(19) United States
(12) Patent Application Publication
(10) Pub. No.: US 2014/0121492 A1
(43) Pub. Date: May 1, 2014

(54) MRI WITH SEPARATION OF DIFFERENT CHEMICAL SPECIES USING A SPECTRAL MODEL

(71) Applicants: Peter Boernert, Hamburg (DE); Holger Eggers, Ellerhoop (DE)

(72) Inventors: Peter Boernert, Hamburg (DE); Holger Eggers, Ellerhoop (DE)

(73) Assignee: KONINKLIJKE PHILIPS N.V., Eindhoven (NL)

(21) Appl. No.: 14/127,037

(22) PCT Filed: Jun. 20, 2012
(86) PCT No.: PCT/IB2012/053101

§ 371 Date: Dec. 17, 2013

(30) Foreign Application Priority Data
Jun. 28, 2011 (EP) 11171643.7

(51) Int. Cl. G01R 33/48; A61B 5/055

(52) U.S. Cl. CPC G01R 33/4828; A61B 5/055
USPC 600/410

(57) ABSTRACT

The invention relates to a method of MR imaging of at least two chemical species having different MR spectra. The method comprises the steps of: generating MR signals of the chemical species by subjecting a portion of a body (10) to an imaging sequence of RF pulses and switched magnetic field gradients, which imaging sequence is determined by a set of imaging parameters (TR, α, TE); acquiring the MR signals; determining a spectral model of at least one of the chemical species, which spectral model is associated with the set of imaging parameters (TR, α, TE); separating signal contributions of the at least two chemical species to the acquired MR signals on the basis of the spectral model; and computing a MR image from the signal contributions of one of the chemical species. Moreover, the invention related to a MR device (1) and to a computer program for a MR device (1).

### TECHNICAL FIELD

The invention relates to the field of magnetic resonance (MR) imaging. It concerns a method of MR imaging of at least two chemical species having different MR spectra. The invention also relates to a MR device and to a computer program to be run on a MR device.

Image-forming MR methods which utilize the interaction between magnetic fields and nuclear spins in order to form two-dimensional or three-dimensional images are widely used nowadays, notably in the field of medical diagnostics, because for the imaging of soft tissue they are superior to other imaging methods in many respects, do not require ionizing radiation and are usually not invasive.

### BACKGROUND OF THE INVENTION

According to the MR method in general, the body of the patient to be examined is arranged in a strong, uniform magnetic field B₀ whose direction at the same time defines an axis (normally the z-axis) of the co-ordinate system on which the measurement is based. The magnetic field B₀ produces different energy levels for the individual nuclear spins in dependence on the magnetic field strength which can be excited (spin resonance) by application of an electromagnetic alternating field (RF field) of defined frequency (so-called Larmor frequency, or MR frequency). From a macroscopic point of view the distribution of the individual nuclear spins produces an overall magnetization which can be deflected out of the state of equilibrium by application of an electromagnetic pulse of appropriate frequency (RF pulse) while the magnetic field B₀ extends perpendicular to the z-axis, so that the magnetization performs a precessional motion about the z-axis. The precessional motion describes a surface of a cone whose angle of aperture is referred to as flip angle. The magnitude of the flip angle is dependent on the strength and the duration of the applied electromagnetic pulse. In the case of a so-called 90° pulse, the spins are deflected from the z axis to the transverse plane (flip angle 90°).

After termination of the RF pulse, the magnetization relaxes back to the original state of equilibrium, in which the magnetization in the z direction is built up again with a first time constant T₁ (spin lattice or longitudinal relaxation time), and the magnetization in the direction perpendicular to the z direction relaxes with a second time constant T₂ (spin-spin or transverse relaxation time). The variation of the magnetization can be detected by means of receiving RF coils which are arranged and oriented within an examination volume of the MR device in such a manner that the variation of the magnetization is measured in the direction perpendicular to the z-axis. The decay of the transverse magnetization is accompanied, after application of, for example, a 90° pulse, by a transition of the nuclear spins (induced by local magnetic field inhomogeneities) from an ordered state with the same phase to a state in which all phase angles are uniformly distributed (dephasing). The dephasing can be compensated by means of a refocusing pulse (for example a 180° pulse). This produces an echo signal (spin echo) in the receiving coils.

In order to realize spatial resolution in the body, linear magnetic field gradients extending along the three main axes are superposed on the uniform magnetic field B₀, leading to a linear spatial dependency of the spin resonance frequency. The signal picked up in the receiving coils then contains components of different frequencies which can be associated with different locations in the body. The signal data obtained via the receiving coils corresponds to the spatial frequency domain and is called k-space data. The k-space data usually includes multiple lines acquired with different phase encoding. Each line is digitized by collecting a number of samples. A set of k-space data is converted to an MR image by means of Fourier transformation.

In MR imaging, it is often desired to obtain information about the relative contribution of different chemical species, such as water and fat, to the overall signal, either to suppress the contribution of some of them or to separately or jointly analyze the contribution of all of them. These contributions can be calculated if information from two or more corresponding echoes, acquired at different echo times, is combined. This may be considered as chemical shift encoding, in which an additional dimension, the chemical shift dimension, is defined and encoded by acquiring a couple of images at slightly different echo times. In particular for water-fat separation, these types of experiments are often referred to as Dixon-type of measurements. By means of Dixon imaging or Dixon water/fat imaging, a water-fat separation can be obtained by calculating contributions of water and fat from two or more corresponding echoes, acquired at different echo times. In general such a separation is possible because there is a known precessional frequency difference of hydrogen in fat and water. In its simplest form, water and fat images are generated by either addition or subtraction of the ‘in phase’ and ‘out of phase’ datasets, but this approach is rather sensitive to main field inhomogeneities. However, such a chemical encoding based separation of different species is not restricted to water/fat species only. Other species with other chemical shifts could also be considered.

High quality water-fat separation with no residual fat signal in water images may be obtained in case complex models of the fat spectrum are incorporated into the water-fat separation process. This has for example been demonstrated for three-point Dixon methods in Yu H, Shimakawa A, McKenzie C A, Brodsky E, Brittain J H, Reeder S B. Multi-echo water-fat separation and simultaneous R2* estimation with multi-frequency fat spectrum modeling. Magn Reson Med 2008; 60:1122-1134.

Another high quality water-fat separation approach using spectral models of the fat spectrum, which consider fat signal dephasing and decay in a two-point Dixon method, has been demonstrated in Eggers H, Brendel B, Duijndam A, Herigault G. Dual-echo Dixon imaging with flexible choice of echo times. Magn Reson Med 2011; 65:96-107.

In particular in time critical applications, two- or three-point methods are preferably used to reduce scan times as much as possible. However, they usually approximate the fat spectrum by a single, dominant peak and thus in general fail to provide an efficient fat suppression. This is because fat is known to comprise multiple spectral peaks. Moreover, the quality of the fat suppression is often suboptimal in the known approaches because they ignore that the contribution from fat to the acquired MR signals substantially varies with the parameters (e.g. repetition time TR, flip angle α, echo times TEᵢ) of the used imaging sequence as well as with the type of the imaging sequence (e.g. spoiled gradient echo sequence, fast spin echo sequence etc.).

### SUMMARY OF THE INVENTION

From the foregoing it is readily appreciated that there is a need for an improved MR imaging technique. It is consequently an object of the invention to provide a method that enhances image quality, notably by achieving a better fat suppression, especially in two- and three-point Dixon methods.

In accordance with the invention, a method of MR imaging of at least two chemical species having different MR spectra is disclosed. The method of the invention comprises the steps of:

generating MR signals of the chemical species by subjecting a portion of a body to an imaging sequence of RF pulses and switched magnetic field gradients, which imaging sequence is determined by a set of imaging parameters;

acquiring the MR signals;

determining a spectral model of at least one of the chemical species, which spectral model is associated with the set of imaging parameters;

separating signal contributions of the at least two chemical species to the acquired MR signals on the basis of the spectral model; and

computing a MR image from the signal contributions of at least one of the chemical species.

According to the invention complex spectral models are employed for signal separation for the different chemical species. As to the details of the spectral modelling it is referred to the above citations.

It has to be noted that it is possible in accordance with the invention that the spectrum of only one of the chemical species is modelled, for example, by a multi-peak spectral model, while another chemical species may simply be modelled by a single-peak spectral model. Consequently, in fact all chemical species are modelled, wherein only one of the models may comprise a multi-peak spectral model.

Further, it has to be noted that the term “chemical species” has to be broadly interpreted as any kind of chemical substance or any kind of nuclei having MR properties. In a simple example, the MR signals of two chemical species are acquired, wherein the chemical species are protons in the “chemical compositions” water and fat. In a more sophisticated example, a multi-peak spectral model actually describes nuclei in a set of different chemical compositions which occur in known relative amounts. In this case, two or more spectral models are used to separate signal contribution from different sets of chemical compositions.

The essential feature of the invention is the provision of a “library” of spectral models, wherein the library includes different spectral models associated with different sets of imaging parameters and/or with different types of imaging sequences. In this way the invention takes into account that the spectrum of one of the chemical species, with which it contributes to the acquired MR signals, substantially varies with the imaging parameters as well as with the sequence type. By taking this variation into account, the invention enables a particularly high quality (water-fat) separation. Moreover, the method of the invention permits a high quality estimation of the main magnetic field inhomogeneity.

The mentioned library of spectral models may comprise a plurality of pre-collected spectral models associated with different sets of imaging parameters stored in a data base. This data base may then serve as a look-up table which is accessed in the signal separation step. The spectral models associated with a set of imaging parameters of the imaging sequence actually used for MR signal generation may be determined by interpolation or extrapolation of the spectral models stored in the library.

It is an important advantage of the invention that the spectra of the different chemical species can be acquired in a separate method step (typically prior to the actual image acquisition procedure) with far higher quality than with the known so-called auto-calibrating approaches which rely solely on the available imaging data for spectral modeling. The spectral modeling can then be based on these pre-collected spectra which results in a particular high-quality signal separation. A further advantage is that complex spectral models can be made available according to the invention even in cases in which the number of echoes is reduced to three or two. In such cases conventional auto-calibrated approaches are no longer able to provide similar information regarding the spectra of the different chemical species as required for high-quality signal separation.

According to a possible embodiment of the invention, the spectral models associated with different sets of imaging parameters may be provided by way of analytical simulation of the respective spectra and/or of the influence of the relevant imaging parameters.

Each spectral model may include resonance frequencies and amplitudes of one or more spectral peaks, phase values and/or relaxation time values. The amplitudes of the spectral peaks determine the relative signal contributions of a chemical species at the different relevant resonance frequencies. The phases describe the de-phasing angle between the spectral peaks and, for example, water protons at a given echo time. Relaxation times may be included to describe the exponential signal decay with echo time. The weights (i.e. the amplitudes of the spectral peaks) and the phases depend on the imaging parameters. Hence, the weights and phases are provided in accordance with the invention for different sets of imaging parameters.

The imaging parameters include the repetition time, the flip angle, and/or at least one echo time of the imaging sequence used for generation of MR signals.

The method of the invention described thus far can be carried out by means of a MR device including at least one main magnet coil for generating a uniform, steady magnetic field B₀ within an examination volume, a number of gradient coils for generating switched magnetic field gradients in different spatial directions within the examination volume, at least one body RF coil for generating RF pulses within the examination volume and/or for receiving MR signals from a body of a patient positioned in the examination volume, a control unit for controlling the temporal succession of RF pulses and switched magnetic field gradients, and a reconstruction unit. The method of the invention can be implemented by a corresponding programming of the reconstruction unit and/or the control unit of the MR device.

The method of the invention can be advantageously carried out on most MR devices in clinical use at present. To this end it is merely necessary to utilize a computer program by which the MR device is controlled such that it performs the above-explained method steps of the invention. The computer program may be present either on a data carrier or be present in a data network so as to be downloaded for installation in the control unit of the MR device.

### BRIEF DESCRIPTION OF THE DRAWINGS

The enclosed drawings disclose preferred embodiments of the present invention. It should be understood, however, that the drawings are designed for the purpose of illustration only and not as a definition of the limits of the invention. In the drawings:

FIG. 1 shows a MR device for carrying out the method of the invention;

FIG. 2 schematically shows MR spectra of fat obtained under varying imaging parameters;

FIG. 3 illustrates a library of fat spectra, stored in a data base as a two-dimensional array according to the invention;

FIG. 4 illustrates a library of fat spectra, stored in a data base as a three-dimensional array according to the invention.

### DETAILED DESCRIPTION OF THE EMBODIMENTS

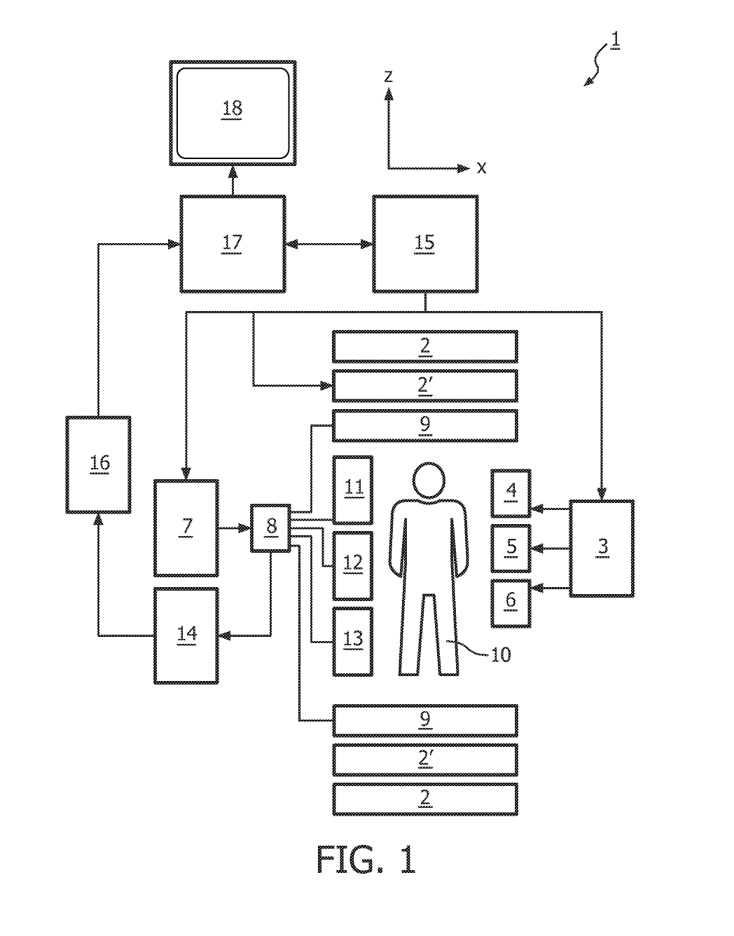

With reference to FIG. 1, a MR device 1 is shown. The device comprises superconducting or resistive main magnet coils 2 such that a substantially uniform, temporally constant main magnetic field B₀ is created along a z-axis through an examination volume. The device further comprises a set of (1^{st}, 2^{nd}, and—where applicable—3^{rd} order) shimming coils 2′, wherein the current flow through the individual shimming coils of the set 2′ is controllable for the purpose of minimizing B₀ deviations within the examination volume.

A magnetic resonance generation and manipulation system applies a series of RF pulses and switched magnetic field gradients to invert or excite nuclear magnetic spins, induce magnetic resonance, refocus magnetic resonance, manipulate magnetic resonance, spatially and otherwise encode the magnetic resonance, saturate spins, and the like to perform MR imaging.

More specifically, a gradient pulse amplifier 3 applies current pulses to selected ones of whole-body gradient coils 4, 5 and 6 along x, y and z-axes of the examination volume. A digital RF frequency transmitter 7 transmits RF pulses or pulse packets, via a send-/receive switch 8, to a body RF coil 9 to transmit RF pulses into the examination volume. A typical MR imaging sequence is composed of a packet of RF pulse segments of short duration which, together with any applied magnetic field gradients, achieve a selected manipulation of nuclear magnetic resonance. The RF pulses are used to saturate, excite resonance, invert magnetization, refocus resonance, or manipulate resonance and select a portion of a body 10 positioned in the examination volume. The MR signals are also picked up by the body RF coil 9.

For generation of MR images of limited regions of the body 10 by means of parallel imaging, a set of local array RF coils 11, 12, 13 are placed contiguous to the region selected for imaging. The array coils 11, 12, 13 can be used to receive MR signals induced by body-coil RF transmissions.

The resultant MR signals are picked up by the body RF coil 9 and/or by the array RF coils 11, 12, 13 and demodulated by a receiver 14 preferably including a preamplifier (not shown). The receiver 14 is connected to the RF coils 9, 11, 12 and 13 via send-/receive switch 8.

A host computer 15 controls the shimming coils 2′ as well as the gradient pulse amplifier 3 and the transmitter 7 to generate any of a plurality of MR imaging sequences, such as echo planar imaging (EPI), echo volume imaging, gradient and spin echo imaging, fast spin echo imaging, and the like. For the selected sequence, the receiver 14 receives a single or a plurality of MR data lines in rapid succession following each RF excitation pulse. A data acquisition system 16 performs analog-to-digital conversion of the received signals and converts each MR data line to a digital format suitable for further processing. In modern MR devices the data acquisition system 16 is a separate computer which is specialized in acquisition of raw image data.

Ultimately, the digital raw image data are reconstructed into an image representation by a reconstruction processor 17 which applies a Fourier transform or other appropriate reconstruction algorithms, such as SENSE or SMASH. The MR image may represent a planar slice through the patient, an array of parallel planar slices, a three-dimensional volume, or the like. The image is then stored in an image memory where it may be accessed for converting slices, projections, or other portions of the image representation into appropriate format for visualization, for example via a video monitor 18 which provides a man-readable display of the resultant MR image.

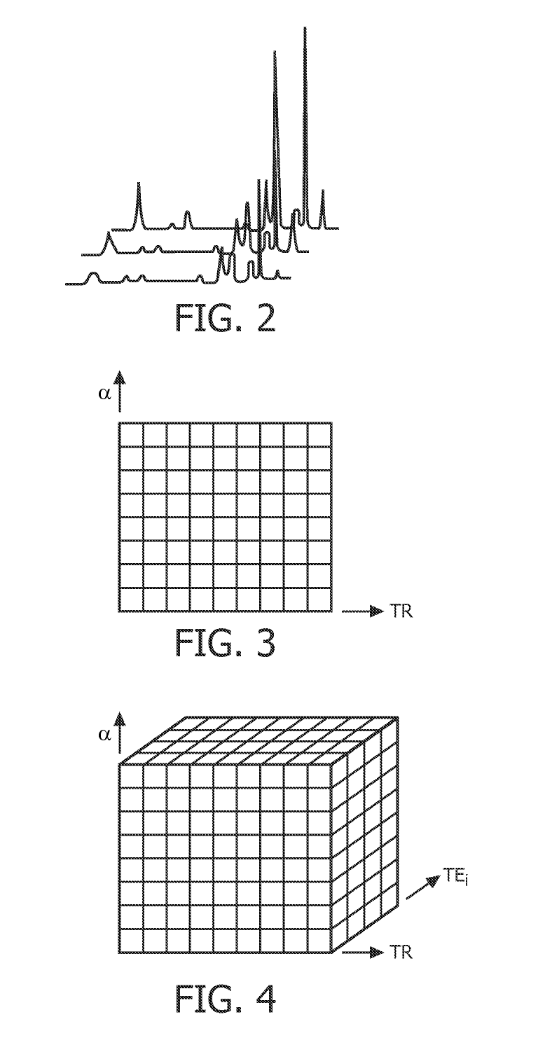

FIG. 2 schematically illustrates MR spectra of fat protons collected under varying imaging parameters (repetition time TR, flip angle α, echo time TE). As can be seen in FIG. 2, the weights, i.e. the amplitudes of the different spectral peaks, substantially vary with the imaging parameters. This variation is considered in accordance with the invention by performing the signal separation in a two- or multi-point Dixon technique on the basis of a spectral model (for example of the fat protons) which is associated with the set of imaging parameters actually used for MR signal acquisition.

According to a first practical embodiment of the invention, chemical shift-encoded three-dimensional gradient-echo imaging is performed for MR signal acquisition with a given repetition time TR and a given flip angle α. The gradient echoes are generated in a RF-spoiled regime to achieve a T₁-weighting. A library of spectral models of fat is used, which has been collected beforehand and thus constitutes prior knowledge. The library includes the amplitudes of the individual spectral peaks, their respective phases and T₂ values. The library contains spectral models for different sets of imaging parameters TR and α, resulting in a matrix as illustrated in FIG. 3. Inter- or extrapolation may be applied when retrieving the amplitudes, phases and T₂ values of the individual spectral peaks for a certain TR and α combination. Alternatively, analytical modeling of the influence of the imaging parameters on the fat spectra may be performed and evaluated on demand. For two-dimensional gradient-echo imaging with poor slice selectivity, resulting in a variation of the flip angle α across the slice, another matrix as shown in FIG. 3 may have to be collected in order to properly reflect the variations in the fat spectra under these conditions.

In another possible embodiment, chemical shift encoded two-dimensional multi-shot fast-spin-echo imaging is performed with a given repetition time TR, inter-echo time TEᵢ, and a refocusing angle α. The fast repetition of the refocusing RF pulses of the imaging sequence can change J-modulation effects, resulting in substantial differences in the fat spectra, namely in T₂ values and also in signal amplitudes. The use of refocusing angles smaller than 180° further results in mixing of different coherence pathways, which are differently exposed to T₁ and T₂ relaxation. This results in an apparent increase in signal lifetime. Therefore, a three-dimensional matrix of spectral models, as illustrated in FIG. 4, is appropriate in this embodiment.

1. Method of MR imaging of at least two chemical species having different MR spectra, the method comprising the steps of:
generating MR signals of the chemical species by subjecting a portion of a body (10) to an imaging sequence of RF pulses and switched magnetic field gradients, which imaging sequence is determined by a set of imaging parameters (TR, α, TE);
acquiring the MR signals;
accessing a library that includes different spectral models associated with different sets of imaging parameters and/or with different types of imaging sequences stored in a data base,
determining from the library a spectral model of at least one of the chemical species, which spectral model is associated with the type of the imaging sequence and/or with the set of imaging parameters (TR, α, TE);
separating signal contributions of the at least two chemical species to the acquired MR signals on the basis of the spectral model; and
computing a MR image from the signal contributions of at least one of the chemical species.

2. Method of claim 1, wherein the spectral model includes resonance frequencies and amplitudes of one or more spectral peaks, phase values and/or relaxation time values.

3. Method of claim 1, wherein the set of imaging parameters (TR, α, TE) includes a repetition time value, a flip angle value, and/or at least one echo time value.

4. Method of claim 1, wherein the MR signals are generated and acquired by means of a two- or multi-point Dixon technique.

5. Method of claim 1, wherein the imaging sequence is a gradient-echo- or spin-echo-type of sequence

6. Method of claim 1, wherein spectral models associated with different sets of imaging parameters (TR, α, TE) are stored in a data base.

7. Method of claim 6, wherein the spectral model associated with the set of imaging parameters (TR, α, TE) of the imaging sequence used for MR signal generation is determined by interpolation or extrapolation of the spectral models stored in the data base.

8. Method of claim 1, wherein spectral models associated with different sets of imaging parameters (TR, α, TE) are provided by way of simulation.

9. MR device for carrying out the method claimed in claim 1, which MR device includes at least one main magnet coil for generating a uniform, steady magnetic field B₀ within an examination volume, a number of gradient coils for generating switched magnetic field gradients in different spatial directions within the examination volume, at least one RF coil for generating RF pulses within the examination volume and/or for receiving MR signals from a body of a patient positioned in the examination volume, a control unit for controlling the temporal succession of RF pulses and switched magnetic field gradients, and a reconstruction unit, and a library that includes different spectral models associated with different sets of imaging parameters and/or with different types of imaging sequences stored in a data base, wherein the MR device is arranged to perform the following steps:
subjecting the portion of the body to an imaging sequence comprising RF pulses and switched magnetic field gradients, which imaging sequence is determined by a set of imaging parameters (TR, α, TE);
acquiring MR signals of at least two chemical species having different MR spectra;
determining from the library a spectral model of at least one of the chemical species, which spectral model is associated with the type of the imaging sequence and/or with the set of imaging parameters (TR, α, TE);
separating signal contributions of the at least two chemical species to the acquired MR signals on the basis of the spectral model; and
computing a MR image from the signal contributions of one of the chemical species.

10. Computer program to be run on a MR device, which computer program comprises instructions for:
generating an imaging sequence comprising RF pulses and switched magnetic field gradients, which imaging sequence is determined by a set of imaging parameters (TR, α, TE);
acquiring MR signals of at least two chemical species having different MR spectra;
accessing a library that includes different spectral models associated with different sets of imaging parameters and/or with different types of imaging sequences stored in a data base,
determining from the library a spectral model of at least one of the chemical species, which spectral model is associated with the type of the imaging sequence and/or with the set of imaging parameters (TR, α, TE);
separating signal contributions of the at least two chemical species to the acquired MR signals on the basis of the spectral model; and
computing a MR image from the signal contributions of one of the chemical species.
